(19) United States
(12) Patent Application Publication
(10) Pub. No.: US 2026/0334895 A1
(43) Pub. Date: Oct. 29, 2026

(54) CONTROL UNIT AND CONTROL METHOD FOR CONTROLLING A MOVABLE COLLIMATOR OF AN X-RAY SYSTEM

(71) Applicant: Siemens Healthineers AG, Forchheim (DE)

(72) Inventors: Thomas WEBER, Hausen, Bayern (DE); Susanne OEPPING, Bubenreuth (DE); Matthias HAAGEN, Nuernberg (DE)

(73) Assignee: Siemens Healthineers AG, Forchheim (DE)

(21) Appl. No.: 19/655,927

(22) Filed: Apr. 23, 2026

(30) Foreign Application Priority Data
Apr. 24, 2025 (DE) 10 2025 115 885.1

(51) Int. Cl. A61B 6/06; A61B 6/00; A61B 6/46; G21K 1/02

(52) U.S. Cl. CPC A61B 6/06; A61B 6/461; A61B 6/467; A61B 6/547; G21K 1/02

(57) ABSTRACT

One or more example embodiments relates to a control unit for controlling a movable collimator of an X-ray system, the rotation of which can be controlled, and wherein the X-ray system comprises a camera which records an area of an imaging detector of the X-ray system. Furthermore, one or more example embodiments comprises a control method, a control facility, and an X-ray system.

### CROSS-REFERENCE TO RELATED APPLICATION(S)

The present application claims priority under 35

U.S.C. § 119 to German Patent Application No. 10 2025 115 885.1, filed Apr. 24, 2025, the entire contents of which are incorporated herein by reference.

### Field

One or more example embodiments relates to a control unit and a control method for controlling a movable collimator of an X-ray system, a control facility for an X-ray system and an X-ray system.

### Related Art

Conventional X-ray imaging systems usually use a collimator, also referred to as a multileaf collimator, to restrict X-ray radiation to an area for examination. To adjust the radiation area or the recording area of an X-ray imaging system, a radiation area is adjusted to an object for examination by adjusting an aperture of the collimator, as a rule via movable metal blades. In addition, the collimator is usually manually rotatable, as a result of which the recording area can also be adjusted. However, as a rule the collimator cannot be rotated 360 °, but has only a limited rotation range of +−45 °. Furthermore, this collimator rotation is usually performed manually by a user and often requires additional optimized manual re-collimation.

### SUMMARY

At present it is therefore very cumbersome and time-consuming to adjust an X-ray imaging system in accordance with an area for examination.

One or more example embodiments provides a control unit and a control method for controlling a movable collimator of an X-ray system, a control facility for an X-ray system and an X-ray system with which the disadvantages described above are avoided. In particular, one or more example embodiments provides a simple and user-friendly method of adjusting a collimator.

### BRIEF DESCRIPTION OF THE DRAWINGS

The invention is explained in more detail with reference to the attached figures and based on the exemplary embodiments. In the various figures, identical components are provided with identical reference characters. The figures are generally not to scale. The diagrams show:

FIG. 1 An X-ray system and a control unit for controlling a movable collimator of the X-ray system,

FIG. 2 An example of a control method according to one or more example embodiments,

FIG. 3 Another example of a control method according to one or more example embodiments, and

FIG. 4 A block diagram for a control method according to one or more example embodiments.

### DETAILED DESCRIPTION

The control unit according to one or more example embodiments of the invention is used to control a movable collimator of an X-ray system, the rotation of which is controllable, and the X-ray system having a camera which records the area of an imaging detector of the X-ray system.

In addition, the control unit according to one or more example embodiments of the invention comprises the following components:
A data interface configured to receive image data from the camera of the X-ray system.
A display unit, for example, an LED display or another screen, configured to display received image data and a control frame,
an input unit, for example, a touch function of the screen, or a stylus which uses this touch function, a mouse, or a keyboard which is configured to detect user actions,
a processing unit which is configured a) to display the control frame on the display unit together with the image data in such a manner that it covers an area over the image data which corresponds to the area onto which X-rays fall when an image is captured by the X-ray system using the current settings of the collimator,
b) to convert detected user actions into a corresponding movement of the control frame and to rotate the control frame in the image plane in response to specified user actions,
c) to additionally convert these user actions into corresponding control commands for controlling the position of the collimator,
a data interface configured to issue control commands for moving the collimator.

The camera of the X-ray system is, for example, an RGB camera, which can be arranged on the collimator. Thus, for example, a video stream recorded by the camera which records or displays an object for examination can be received by the data interface and displayed on the display unit. Even if a 3D camera can certainly be advantageous, a simple 2D camera is essentially sufficient for one or more example embodiments. It is preferable that the image data is or comprises a video stream or a series of consecutive frames so that the current position of the object can be displayed in real time.

Together with these images, the control frame is displayed on the display unit, thus for example, this control frame together with a video stream from the camera. The control frame covers an area corresponding to the area onto which X-rays fall when an image is acquired by the X-ray system using the current settings of the collimator. This is easy to accomplish. The current position of the collimator, including its aperture, must simply be determined. For controllable collimators, this data is usually available already and so it can be read in directly.

Optimally, the control frame should already be positioned over the displayed object. However, as this depends on the position of the collimator, this is not yet the case usually. A means of automated presetting is described below. However, it is not necessary for the object for examination to be selected by the control frame (i.e., by the collimator setting) as the control frame is preferably intended to serve as a control element for optimal adjustment of the collimator.

Corresponding user actions specified or entered by the user rotate the control frame in the image plane, i.e., it rotates about the surface normal to the image plane. For example, a mouse pointer can be moved onto the control frame, and the control frame can be selected with a mouse click and the control system thus activated. By moving the mouse, the control frame is then rotated, as is already known, for example, in CAD programs with regard to the rotation of selected objects. However, the control frame can also be displayed on a touchscreen and moved using gestures on the touchscreen.

It is certainly possible to also control the location (position and orientation) of the collimator using a control unit, and to adjust the control frame in accordance with this control unit. However, what is important for one or more example embodiments is that the control frame (at least also) serves as a control element for the collimator.

User actions therefore move the control frame and are also converted into corresponding control commands for controlling the position of the collimator, in the case of specified user actions in which the control frame is rotated in the image plane, corresponding control commands for rotating the collimator being generated (directly or indirectly via the position of the control frame), or control of the collimator being reflected in a movement of the control frame.

The data interface is used to issue control commands to the collimator. Such data interfaces are known in the prior art.

A control method according to one or more example embodiments is used to control a movable collimator of an X-ray system, the rotation of which can be controlled, and the X-ray system having a camera which records the area of an imaging detector of the X-ray system, with a control unit according to one or more example embodiments. The control method comprises the following steps:
Provision of image data from the camera of the X-ray system,
Display of the provided image data on the display unit of the control unit,
Detection of user actions by the input unit of the control unit,
Display of a control frame on the display unit together with the image data, such that it covers an area over the image data corresponding to the area onto which X-rays fall with the current settings of the collimator during the acquisition of an image by the X-ray system,
Conversion of detected user actions into a corresponding movement of the control frame and rotation of the control frame in the image plane upon receipt of specified user actions,
Conversion of these user actions into corresponding control commands for controlling the collimator and preferably also for controlling the position of the imaging detector, the control commands for controlling the collimator in particular also including controlling of its blades,
Issuing of control commands for moving the collimator and, if present, also for controlling the imaging detector.

Essentially, the control method, in which the control frame represents a virtual control element for the collimator, has already been described above in the context of the control unit. The provision of image data, the output of control data, and the display of provided image data on a display unit are already known in the prior art.

User actions can be gestures on a touchscreen or a touchpad or involve the use of a data glove as an input unit. However, they can also be movements and clicks of a mouse as an input unit or movements of a joystick as an input unit. Theoretically, a keyboard could also be used as an input unit. The user actions would then be keystrokes.

The control frame is preferably displayed in such a manner that it overlays the image data, while the image data remains visible inside and around the frame. For example, a simple rectangle can be displayed on the screen, possibly with graphical representations of control elements.

The image can be displayed directly, such that the position of the collimator is first determined; based on this, it is calculated onto which area the X-rays will fall when the X-ray system captures an image using the current settings of the collimator; then it is determined which image area in the display corresponds to this (illuminated) area, and the control frame is positioned precisely within this image area. However, the control frame cannot be displayed until after the automated presetting of the collimator. To do this, the position of the object can first be determined, then the collimator can be adjusted according to the determined position, and thereafter the control frame can be displayed, as described above, according to the adjusted position of the collimator.

If the control frame is displayed (and, if applicable, selected for an action), then detected user actions can be translated into a corresponding movement of the control frame. At any rate, the control frame rotates in the image plane upon receiving specified user actions. However, it is also preferable that the control frame can be moved to center the object. Furthermore, it is preferable that the size of the control frame can be adjusted, involving a corresponding adjustment of the collimator aperture. This enables the image detail to be selected in such a manner that during a recording, only the desired object (or an area of the body) is irradiated using X-rays.

The user actions are also converted into corresponding control commands for controlling the collimator and preferably also for controlling the position of the imaging detector, the control commands for controlling the collimator in particular also including the control of its blades. It is possible here that the control frame serves to control the collimator, that the collimator controls the control frame, or both; that, on the one hand, the control frame can be moved and the position of the collimator is thus controlled, but conversely, the collimator can also be controlled via its control console and the control frame displays its position. It should be noted that the conversion of user actions into corresponding control commands for controlling the collimator can also be performed indirectly by applying the user actions to the positioning of the control frame and then deriving the control commands from the positioning of the control frame (which is determined by the user actions).

With regard to controlling the imaging detector, some systems also allow the detector to be rotated by a motor. If the control frame is rotated, the imaging detector could also be rotated accordingly, so that the object is already aligned accordingly in the image.

A control facility (also referred to as a control system) according to one or more example embodiments for an X-ray system comprises a control unit according to one or more example embodiments. Alternatively or additionally, it is configured to perform a control method according to one or more example embodiments.

An X-ray system according to one or more example embodiments comprises a control facility according to one or more example embodiments.

The method may also be referred to as a computer-implemented method. One or more example embodiments may, in particular, be implemented in the form of a processing unit with suitable software. The processing unit may, for example, comprise one or more cooperating microprocessors or the like. In particular, it may be implemented in the form of suitable software program modules in the processing unit. The advantage of largely software-based implementation is that even processing units used hitherto can be easily retrofitted via a software or firmware update to operate according to one or more example embodiments. In this respect, the object is also achieved by a corresponding computer program product with a computer program which can be loaded directly into a storage facility (also referred to as a storage system) of a processing unit, with program sections for performing all the steps of the method according to one or more example embodiments when the program is executed in the processing unit. In addition to the computer program, such a computer program product may, if applicable, include additional components such as, for example, documentation and/or additional components, including hardware components such as, for example, hardware keys (dongles, etc.) for using the software.

A computer-readable medium, for example a memory stick, a hard disk or any other portable or built-in data carrier may be used to transfer data to the processing unit and/or to store it on or in the processing unit on which the program sections of the computer program which are readable and executable by a processing unit are stored.

Further, particularly advantageous embodiments and developments of the invention will emerge from the dependent claims and the following description, it being possible for the claims in one claim category to also be developed analogously to the claims and descriptions in another claim category and, in particular, individual features of different exemplary embodiments or variants may also be combined to form new exemplary embodiments or variants.

A preferred control unit comprises a data interface configured to receive position parameters relating to the position of the collimator and its current angle of rotation, and preferably also relating to the position of its blades. This is highly advantageous if the collimator can determine and output its own parameter values relating to the current position. Otherwise, the position of the collimator could also be determined using external elements and made available to the method. It is preferable that the processing unit is configured to display the control frame on the display unit in accordance with received parameters of the collimator.

A preferred control unit is characterized in that the control frame is correlated with the frame of an image recording and/or can be defined, preferably in that at least one frame element of the control frame, in particular one corner or one edge, is correlated with a corresponding frame element of the output image, in particular an upper corner or edge. It should be noted that this does not necessarily involve the movement of an imaging detector. In this way, it is also possible for an image detail in the recorded image to be defined and for this image detail to be mapped to a predetermined, preferably rectangular area using software. For example, one edge of the control frame can be defined as the top edge and, if necessary, specifically marked, for example, colored. If the control frame is now rotated, it is possible that an image detail will lie at an angle on the imaging detector. If an image is now captured, the defined correlation (the top edge of the control frame) automatically crops a rectangular image from the entire image data of the imaging detector which corresponds to the control frame and in which the top is where the top edge of the control frame was located.

It is preferable that the processing unit is configured to perform such a correlation in response to a specified user action, in particular when a user selects a particular frame element, for example, by double-clicking. Rotating an image (automatically) in accordance with the rotation of the collimator is also preferable. The orientation of the image then preferably corresponds to the orientation of the collimator, the top edge of the image being predetermined in accordance with the correlation.

It is preferable that the control unit is configured to optimize the position of the object in the image. This refers to a readjustment of the position of the object in the image. For example, a hand can be rotated slightly so that it is optimally aligned in the image. This facilitates better post-processing. Readjustment can take place, for example, by comparing the image edges (edge areas which do not depict the object) of opposite sides of the control frame with one another and centering the object by moving the control frame so that the opposite image edges are equal in size.

The control frame preferably has virtual grip elements with which a user can interact virtually, for example, via a mouse pointer or a touchscreen, and can move the frame in this way. Such elements are known in the prior art, for example in CAD programs.

A preferred control unit is characterized in that, after capturing an image which is correlated with the control frame, the processing unit is configured to use this correlation as additional information for image processing, preferably for determining an optimal angle of rotation for the automatic rotation of an object image, and/or for mapping the pixels of the captured image onto a pixel grid corresponding to the control frame. It should be noted here that an imaging detector customarily has a rectangular structure consisting of image rows and image columns. If an image detail is tilted on the imaging detector, calculations must be performed to map the image detail back onto a grid of image rows and image columns. This is done via image processing. However, to do so, it requires the position of the control frame which is transmitted to it in this embodiment.

A further difficulty is to determine the preferred direction or orientation of the object to be irradiated, or of the image to be captured. This is particularly difficult in free scans, i.e., in recordings in which a mobile detector is positioned freely in space and not in a specific detector tray.

Preferably, the detection of a preferred orientation is based on machine learning. A neural network specifically trained for this purpose is often used. However, the neural network requires extensive training to determine the correct angle of rotation of the X-ray image. To this end, training data in the form of images can be obtained and annotated with basic truth (e.g., markers indicating the preferred orientation). All variants and preferences of the respective user should also be taken into account here. National or local requirements and preferences can also lead to different preferred orientations. For example, an image of an organ may be requested, for example rotated by 90 °or 180 °, so that, for example, the X-ray image of a hand faces upward or downward.

In a preferred control unit, the processing unit is configured to preset the collimator based on a user action or based on automated detection (and then to display the control frame accordingly). It is preferable that this takes place by
a user or an automated detection system marking an area on the display unit,
a bounding box being generated around the marked area,
control commands for moving the collimator being issued which are configured in such a manner that, after adjustment according to the control commands, the collimator covers an area corresponding to the area of the bounding box, and—a corresponding control frame is then displayed on the display unit.

Marking can be performed, for example, by a user circling or shading an object. An automated system can select an area via image segmentation or region growing. It is then very easy to automatically generate a rectangular frame around such an area. For example, an area of a body can be selected by double-clicking on the display unit. A machine learning model trained accordingly then recognizes the selected body part and marks it. A bounding box is then generated around this area, and the collimator is adjusted in accordance with this bounding box. The bounding box essentially serves as a control frame and can be displayed directly as a control frame. It should be noted that the generation of the bounding box does not necessarily have to include the display of the bounding box, although this is preferable.

In a preferred control unit, the processing unit is configured to map the limits of the movement of the collimator, in particular, the limits of its angle of rotation, onto the movement of the control frame. As already mentioned above, as a rule a collimator cannot be rotated through the full 360 °. The control frame should also reflect this, so that it cannot be rotated further in the display unit above the displayed image than the collimator, ensuring that it always displays the “illuminated” area. It is therefore preferable that the processing unit is configured in such a manner that, when the collimator reaches the limit of its movement, the control frame on the display unit cannot be moved beyond that limit. The position of the collimator and the control frame should always correspond to one another.

A preferred control unit is characterized in that, in the event that the collimator and the camera move together within the X-ray system, rotating the collimator causes the image data to be displayed on the display unit rotated in the opposite direction, so that the displayed image remains stationary on the display unit. This has the advantage that a user is better able to orientate themselves. Thus, if the collimator with the camera 20 °rotates to the left, the image would also rotate 20 °to the left as the camera is also moving. If the image is rotated 20 °to the right using software during this process, it appears to remain on the display unit. It is preferable that an image parallax and/or an arc movement of the camera is compensated for by a corresponding shift of the image.

A preferred control unit comprises a data interface configured to receive position parameters relating to the position of the imaging detector. The data is preferably determined by a sensor. The imaging detector may, for example, incorporate a 6- or9 -axis inertial sensor which transmits the tilt angles of the detector relative to gravity to the system. Other methods for determining the position of the detector are acoustic methods (for example, via ultrasound) or electromagnetic positioning methods (for example, via a magnetic field). Based on the position of the control frame, it is preferable that the processing unit is configured to also generate corresponding control commands for controlling the position of the imaging detector, such that the control frame fully overlaps the imaging detector, and preferably also such that the rows of the imaging detector are aligned parallel to two edges of the control frame, preferably with its upper and lower edge. No time-consuming adjustment of the image area is required as a result.

In a preferred control unit, the processing unit is configured to alter the dimensions of the control frame in response to specified user actions and to issue corresponding control commands which control the blades of the collimator accordingly. If the frame is enlarged, the aperture of the collimator also increases; if the frame is reduced, the aperture of the collimator also decreases. It is preferable that the processing unit is configured to automatically determine a bounding box around the object enclosed by a control frame prior to a rotation of the control frame, and after a rotation of the control frame, to adjust the blades of the collimator in accordance with the bounding box such that the bounding box is completely contained within the rotated frame. This can also be performed as part of the presetting described above. The size of the bounding box can be specified if the blades are not to be moved, but a bounding box can also be generated such that a movement of the blades is also taken into account.

In one embodiment of the control method, it is preferable that, after a specified user action, the control frame is correlated with and/or defined relative to the frame of an output image (see above), this correlation being used in particular as additional information for image processing. Alternatively or additionally, the control frame may be displayed inertially on the display unit in accordance with this user action.

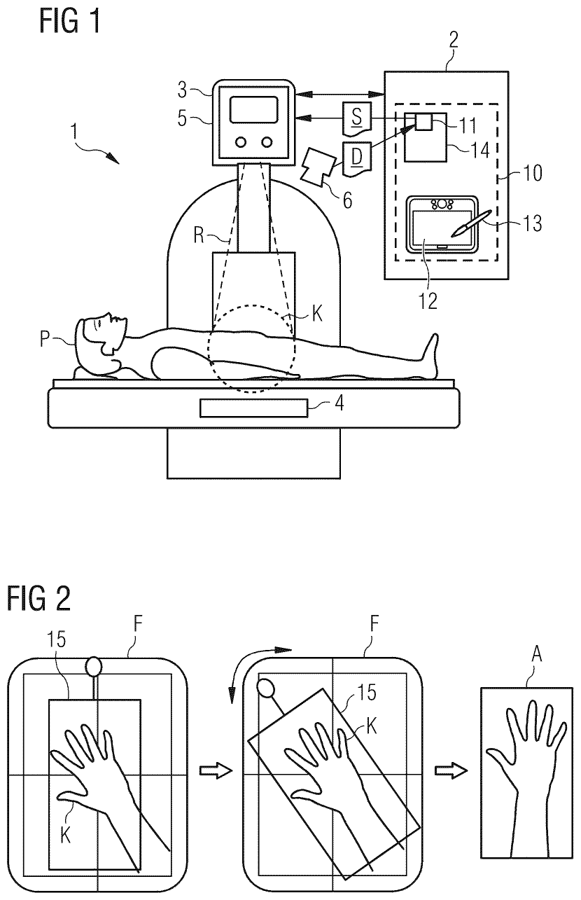

FIG. 1 shows an X-ray system 1 and a control unit 10 for controlling a movable collimator 5 of the X-ray system 1. A body part K of a patient P is irradiated with X-rays R from a source of radiation 3 as an object K for examination. A collimator 5 is used to limit the X-rays onto the body part K. The patient P is located between the source of radiation 3 and an imaging detector 4 of the X-ray system, or the patient P lies on the imaging detector 4. In addition, a camera 6 captures image data D, for example, a live video, of an area of the imaging detector 4. The captured image data D is then received by a data interface 11. The image data D is displayed on a display unit 12, in this example, a touchscreen on a tablet computer. The data interface 11 is integrated into a processing unit 14. Via an input unit 13—in this case, a stylus for the touchscreen—of the control unit 10, corresponding user actions can be entered and recorded. Via a control facility 2 comprising the control unit 10, control commands S can be issued to move the collimator 5.

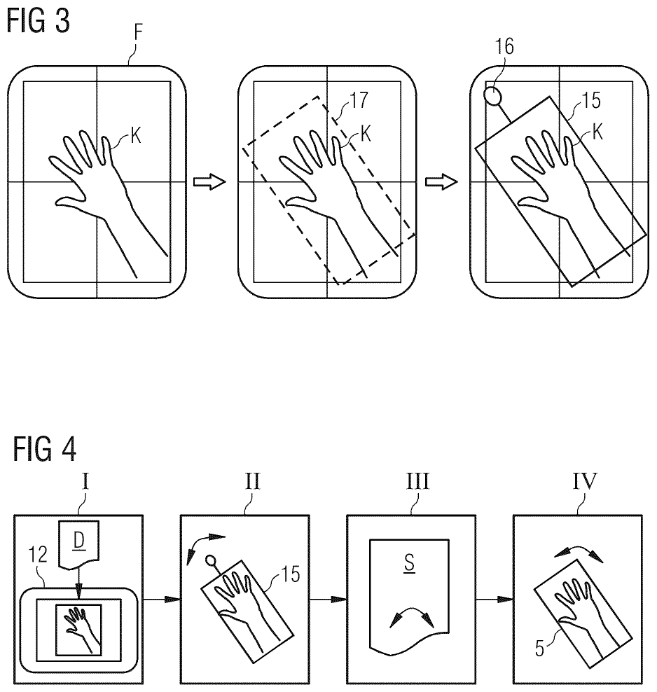

FIGS. 2 and 3 illustrate the functions of the control unit 10, in particular its processing unit 14.

The processing unit 14 of the control unit 10 is configured to display the control frame 15 together with the image data D on the display unit 12 in such a manner that, via the image data D, it overlays an area corresponding to the area onto which X-rays R fall when an image is captured by the X-ray system 1 using the current settings of the collimator 5. This is illustrated, for example, in the middle image of FIG. 2.

When a user touches the touchscreen using the stylus as the input unit 13, this user action is detected. Detected user actions are converted into a corresponding movement of the control frame 15 by the processing unit 14 and the control frame 15 is rotated in the image plane in response to specified user actions. This is symbolized by the double arrow in the middle image of FIG. 2. In addition, the processing unit 14 generates corresponding control commands S from these user actions to control the position of the collimator 5. This rotates in accordance with the control frame 15.

Control commands S for movement of the collimator 5 are issued via a data interface 11 (in this example, it is generally used for data communication).

In this example, the data interface 11 also receives position parameters relating to the position of the collimator 5 and its current angle of rotation as well as preferably also to the position of its blades. Here, the processing unit 14 is configured to display the control frame 15 on the display unit 12 in accordance with the received parameters of the collimator 5.

In this example, the processing unit 14 is configured to transfer the limits of the movement of the collimator 5, in particular, the limits of its angle of rotation, to the movement of the control frame 15. When a limit of the movement of the collimator 5 is reached, the control frame 15 cannot be moved beyond that limit on the display unit 12.

In this example, the imaging detector 4 is arranged fixedly in the X-ray system 1. If it were movable, the control unit 10 could be configured to receive position parameters relating to the position of the imaging detector 4 via a data interface 11. Based on the position of the control frame 15, the processing unit 14 would then be configured to generate additional corresponding control commands S for controlling the position of the imaging detector 4, which cause the control frame 15 to overlap the imaging detector 4 over its entire surface and, preferably, also to align the rows of the imaging detector 4 parallel to two edges of the control frame 15, preferably its upper and lower edges.

The processing unit 14 could also be configured to alter the dimensions of the control frame 15 in response to specified user actions and to issue corresponding control commands S which control the blades of the collimator 5 accordingly.

FIG. 2 shows a body part K, in this case a hand, in a field of view F of the control unit 10 described in FIG. 1. This is a sequence of images from the camera 6. This field of view F is captured by the camera 6 and displayed on the display unit 12. To display an image recording A in a preferred orientation and to adapt a radiation area to the body part K, a control frame 15 is first selected or displayed in such a manner that it covers an area of the field of view corresponding to the area onto which X-rays R are intended to fall with the current settings of the collimator 5 during the acquisition of an image by the X-ray system 1.

In the image on the left, the hand has just been placed in the field of view F, and a control frame 15 has been superimposed over the image of the hand. It can be seen that it does not align with the position of the hand because the collimator 5 has not yet been optimally adjusted.

In the middle image, the control frame 15 is rotated as indicated by the double arrow. As a result, the collimator 5 rotates accordingly.

In this example, the control frame contains a grip element 16, which represents a virtual pivot point for rotating the control frame. At the same time, it can indicate the location of the top edge of the control frame 15. In this example, the control frame 15 is correlated with the frame of an image recording A, and the top edge of the control frame simultaneously indicates the top edge of the future image recording A. The processing unit 14 is configured here to perform such a correlation in response to a specified user action, and also to perform an image rotation corresponding to the rotation of the collimator 5.

The image on the right shows the resulting image recording A. The area illuminated by X-rays R (see FIG. 1) was defined by the collimator 5 and the display of image recording A was rotated according to the position of the control frame 15. The processing unit 14 is thus configured here to use this correlation, after capturing an image which is correlated with the control frame 15, as additional information for determining an optimal angle of rotation for the automatic rotation of an object image.

FIG. 3 shows the process in a preferred embodiment in which the processing unit 14 is configured to preset the collimator 5 based on a user action or based on automated detection. A sequence of images from the camera 6 is also shown here.

Here too, a hand is placed in the field of view F on the left, but no control frame 15 is superimposed over the image of the hand as shown in FIG. 2. However, for a better understanding, it is possible to imagine that the collimator 5 is aligned as indicated by the control frame 15 in the image on the left in FIG. 2.

In the middle image, a user or an automated detection system has marked the hand on the display unit 12, and the processing unit 14 has generated a bounding box 17 around the marked area. This bounding box 17 may be displayed or used solely for further actions; it is therefore shown as a dashed line.

Control commands S are now issued to move the collimator 5 in such a manner that, after being adjusted according to the control commands S, the collimator covers an area corresponding to the area of the bounding box 17.

The image on the right shows a corresponding control frame 15 which is displayed on the display unit 12 using the control commands S after adjustment of the collimator 5, and which covers the hand well.

FIG. 4 shows a block diagram for a control method according to one or more example embodiments for controlling a movable collimator 5 of an X-ray system 1 according to FIG. 1.

In Step I, image data D from the camera 6 of the X-ray system 1 is provided and displayed on the display unit 12 of the control unit 10.

In Step II, user actions are detected by the input unit 13 of the control unit 10, for example, movements of the stylus of the tablet computer in FIG. 1 on its touchscreen, and a control frame 15 displayed on the display unit 12 is moved accordingly in response to a user action.

In Step III, the user actions are converted into corresponding control commands S for controlling the collimator 5. Control commands S for controlling the position of the imaging detector 4 may also be generated.

In Step IV, the control commands S are issued to move the collimator 5 and, if present, also to control the imaging detector 4.

Lastly, it should be noted once again that the invention described in detail above consists solely of exemplary embodiments which may be modified in various ways by a person skilled in the art without departing from the scope of the invention. Furthermore, the use of the indefinite article “a” or “an” does not preclude the possibility that the relevant features may be present multiple times. Likewise, terms such as “unit” do not preclude the possibility that the relevant components consist of several interacting subcomponents which may also be spatially distributed. The term “a number” is to be interpreted as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as an data processing system) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A control unit configured to control a movable collimator of an X-ray system including a camera configured to record an area of an imaging detector of the X-ray system, the control unit comprising:
a data interface configured to receive image data from the camera of the X-ray system;
a display configured to display the received image data and a control frame;
an input unit configured to detect user actions;
a processing unit configured to,
display the control frame on the display with the image data such that the control frame overlays an area of the image data corresponding to an area onto which X-rays fall when the X-ray system captures an image using current settings of the collimator,
convert detected user actions into a corresponding movement of the control frame and rotate the control frame in an image plane in response to specified user actions,
convert the detected user actions and the specified user action into corresponding control commands to control a position of the collimator; and
a data interface configured to issue control commands to move the collimator.

2. The control unit of claim 1, wherein
the data interface is configured to receive position parameters regarding the position of the collimator and a current angle of rotation of the collimator, the position parameters regarding a position of blades of the collimator, and
the processing unit is configured to display the control frame on the display in accordance with the position parameters.

3. The control unit of claim 1, wherein
the control frame is at least one of correlated or definable with a frame of an image recording, by correlating at least one frame element of the control frame with a corresponding frame element of an output image, and
the processing unit is configured to perform the correlation upon a specified user action, and perform an image rotation corresponding to the rotation of the collimator.

4. The control unit of claim 3, wherein the processing unit is configured to use the correlation as information for image processing after capturing the image which is correlated with the control frame, and, based on the information for image processing, to determine an optimal angle of rotation for an automatic rotation of an object image and to map pixels of the captured image onto a pixel grid corresponding to the control frame.

5. The control unit of claim 1, wherein
the processing unit is configured to preset the collimator based on a particular user action or based on automated detection,
an area on the display is marked by a user or an automated detection system,
a bounding box is generated around the marked area,
control commands for moving the collimator are issued, which are configured such that, after being adjusted in accordance with the control commands, the collimator covers an area corresponding to the area of the bounding box, and
a corresponding control frame is displayed on the display thereafter.

6. The control unit of claim 1, wherein
the processing unit is configured to transfer limits of the movement of the collimator to the movement of the control frame, and
upon reaching a limit of the movement of the collimator, the control frame cannot be moved beyond the limit on the display.

7. The control unit of claim 1, wherein
in an event that the collimator and the camera move together in the X-ray system when the collimator is rotated, the image data is displayed on the display rotated accordingly in an opposite direction, such that the image represented by the image data remains stationary on the display, and
at least one of an image parallax and/or an arc movement of the camera is compensated for by a corresponding shift of the image.

8. The control unit of claim 1, wherein
the data interface is configured to receive position parameters regarding a position of the imaging detector, the processing unit is configured to generate corresponding control commands for controlling the position of the imaging detector based on the position of the control frame, such that the control frame overlaps an entire surface of the imaging detector, and such that rows of the imaging detector are aligned parallel to upper and lower edges of the control frame.

9. The control unit of claim 1, wherein
the processing unit is configured to change dimensions of the control frame in response to specified user actions and to issue corresponding control commands which control blades of the collimator, and
the processing unit is configured to, prior to a rotation of the control frame, to automatically determine a bounding box around an object surrounded by the control frame and, after a rotation of the control frame, to adjust blades of the collimator in accordance with the bounding box such that the bounding box is completely contained within the rotated frame.

10. A control method for controlling a movable collimator of an X-ray system, the X-ray system including a camera which records an area of an imaging detector of the X-ray system, the method comprising:
providing image data from the camera of the X-ray system;
displaying the provided image data on a display of a control unit;
detecting user actions by an input unit of the control unit;
displaying a control frame on the display together with the image data such that it covers an area over the image data corresponding to an area onto which X-rays fall with the settings of the collimator during an acquisition of an image by the X-ray system;
converting detected user actions into a corresponding movement of the control frame and rotation of the control frame in an image plane upon receipt of specified user actions;
converting the detected user actions and the specified user actions into corresponding control commands for controlling the collimator and for controlling a position of the imaging detector, wherein the control commands for controlling the collimator include control commands for controlling blades of the collimator; and
issuing control commands for moving the collimator and for controlling the imaging detector.

11. The control method of claim 10, wherein after a specified user action at least one of
the control frame is at least one of correlated with or defined relative to the frame of an output image, wherein the correlation is used as additional information for image processing, or
initially, the control frame is displayed on the display in accordance with this user action the specified user action.

12. A control facility for an X-ray system, the control facility comprising the control unit of claim 1.

13. An X-ray system comprising the control facility of claim 12.

14. A non-transitory computer programming product, comprising instructions which, when executed by a computer, cause the computer to the method of claim 10.

15. A non-transitory computer-readable storage medium, comprising instructions which, when executed by a computer, cause the computer to perform the method of claim 10.
